(19) United States
(12) Patent Application Publication
(10) Pub. No.: US 2006/0217760 A1
(43) Pub. Date: Sep. 28, 2006

(54) Multi-strand septal occluder

(76) Inventors: David R. Widomski, New York, NY (US); John A. Wright JR., Lexington, MA (US)

Correspondence Address: KIRKPATRICK & LOCKHART NICHOLSON GRAHAM LLP, STATE STREET FINANCIAL CENTER, ONE LINCOLN STREET, BOSTON, MA, 02111-2950, US

(21) Appl. No.: 11/377,010

(22) Filed: Mar. 16, 2006

Related U.S. Application Data
(60) Provisional application No. 60/662,780, filed on Mar. 17, 2005.

(51) Int. Cl. A61B 17/08

(52) U.S. Cl. 606/213

(57) ABSTRACT

Disclosed herein are devices and methods for occluding intracardiac defects, such as a patent foramen ovale (PFO). The devices according to the invention have various features to improve flexibility and to enhance conformability of the device to the defect, including the incorporation of braided or multi-stranded wire. The invention also contemplates methods of making these devices.

### CROSS-REFERENCE TO RELATED APPLICATIONS

This application claims priority to and the benefit of U.S. Provisional Patent Application Ser. No. 60/662,780, filed on Mar. 17, 2005, the entire disclosure of which is incorporated by reference herein.

### TECHNICAL FIELD OF THE INVENTION

The invention generally relates to devices and related methods for treating intracardiac defects. More particularly, the invention provides an intracardiac occluder for the percutaneous closure of intracardiac defects, including patent foramen ovale (PFO).

### BACKGROUND OF THE INVENTION

The human heart is divided into four compartments or chambers. The left and right atria are located in the upper portion of the heart and the left and right ventricles are located in the lower portion of the heart. The left and right atria are separated from each other by a muscular wall, the intra-atrial septum, while the ventricles are separated by the intraventricular septum.

Either congenitally or by acquisition, abnormal openings, holes, or shunts can occur between the chambers of the heart or the great vessels, causing blood to flow therethrough. Such deformities are usually congenital and originate during fetal life when the heart forms from a folded tube into a four chambered, two unit system. The deformities result from the incomplete formation of the septum, or muscular wall, between the chambers of the heart and can cause significant problems. Ultimately, the deformities add strain on the heart, which may result in heart failure if they are not corrected.

One such deformity or defect, a patent foramen ovale, is a persistent, one-way, usually flap-like opening in the wall between the right atrium and left atrium of the heart. Since left atrial pressure is normally higher than right atrial pressure, the flap typically stays closed. Under certain conditions, however, right atrial pressure exceeds left atrial pressure, creating the possibility for right to left shunting that can allow blood clots to enter the systemic circulation. This is particularly worrisome to patients who are prone to forming venous thrombus, such as those with deep vein thrombosis or clotting abnormalities.

Nonsurgical (i.e., percutaneous) closure of a PFO, as well as similar intracardiac defects such as atrial septal defects, ventricular septal defects, and closure of left atrial appendages, is possible using a variety of mechanical closure devices. These devices, which allow patients to avoid the potential side effects often associated with standard anticoagulation therapies, typically consist of a metallic structural framework that is combined with a synthetic or biological tissue scaffold material. The support structure of the septal occluder is often stiff and rigid, lacking flexibility to conform with septal defects, resulting in trauma to surrounding tissues, chronic inflammation, residual leaks and reduced rates of defect closure.

### SUMMARY OF THE INVENTION

The present invention provides a device for occluding intracardiac defects, as well as a method for making the device. The device includes a flexible and resilient support structure coupled with a scaffold material to create a flexible septal occluder. The support structure comprises a variety of modifications that enhance the flexibility of the device.

In one aspect, the invention is directed to a flexible septal occluder. In one embodiment of this aspect of the invention, a flexible septal occluder comprises a first portion and a second portion joined by a central body portion. The first portion comprises a plurality of arms with a flexural point disposed on at least one of the arms. The second portion also comprises a plurality of arms. At least one of the arms on the first portion comprises a plurality of strands forming a bundle of strands interrupted at the flexural point by a gap in strands. Further features of this embodiment include the following. For example, the embodiment may feature more than one flexural point disposed on at least one arm of the first portion. At least one flexural point may also be disposed on each arm of the first portion. In addition, a plurality of strands comprising the arm may run parallel to one another along the lengthwise axis of the arm. The flexible septal occluder may also comprise at least one flexural point disposed on at least one arm of the second portion. Additional features may include one or more flexural points disposed on the central body portion of the flexible septal occluder. The plurality of strands comprising the central body portion may also run parallel to one another along the lengthwise axis of the central body portion.

In another embodiment of this aspect of the invention, the flexible septal occluder comprises a first portion comprising a plurality of arms. At least one of said arms comprises a plurality of strands forming a bundle of strands comprising a first loop and a second loop. The first portion is joined by a central body portion to a second portion comprising a plurality of arms. The plurality of strands comprises a first loop and a second loop with the first loop comprising a different diameter than said second loop. Further features of this embodiment include the following. For example, the first loop and the second loop may be adjacent. The diameter of the first loop may have the same diameter as the diameter of another loop disposed on the first portion, while a further feature requires that the first loop and the another loop are adjacent. According to another feature of the embodiment, the second portion of the flexible septal occluder comprises a plurality of strands forming a bundle of strands comprising a first loop and a second loop. An additional feature may include at least one of the plurality of arms of the first portion or the second portion comprising a coil, while a further feature includes a central body portion or both the central body portion and at least one of the plurality of arms comprising a coil.

In yet another embodiment of this aspect of the invention, the flexible septal occluder comprises a first portion comprising a plurality of arms, a second portion comprising a plurality of arms, and a central body portion. The first and second portions are joined by the central body portion. The central body portion comprises a plurality of strands forming a bundle of strands forming a first loop and a second loop. The first loop of the central body portion comprises a different diameter than the second loop. Further features of this embodiment include the following. For example, the first loop and the second loop of the central body portion may be adjacent. The embodiment may also feature the central body portion comprising another loop with the same diameter as the first loop. According to another feature of the embodiment, the first loop and the other loop may be adjacent. An additional feature may include at least one of the plurality of arms of the first portion or the second portion comprising a coil, while a further feature includes a central body portion or both the central body portion and at least one of the plurality of arms comprising a coil.

In another embodiment of this aspect of the invention, a flexible septal occluder comprises a first portion comprising a plurality of arms having a plurality of strands, a second portion comprising a plurality of arms having a plurality of strands, and a central body portion comprising a plurality strands. One of the strands of the first, second, or central body portions comprises a non-circular cross-section. Further features of this embodiment include the following. For example, the non-circular cross-section may be triangular, but may also be selected from the group consisting of a square, a rectangular, a triangular, a hexagonal, an elliptical, and a rhomboidal cross-section. As an additional feature, all of the strands of the first portion, second portion or central body portions may comprise the same non-circular cross-section, whereas alternatively, the embodiment may feature at least one of said first portion, second portion, or central body portions comprising at least two strands, with each of the two strands having different non-circular cross-sections.

In another embodiment of this aspect of the invention, the flexible septal occluder comprises a first portion comprising a plurality of arms, a second portion comprising a plurality of arms, and a central body portion comprising a coil. As a further feature, at least a portion of one of the plurality of arms of the first portion or the second portion of the flexible septal occluder comprises a coil.

A further aspect of the invention is a method for making a flexible septal occluder. The method comprises providing a first portion comprising a plurality of arms, said arms comprising a first wire and a second wire, and providing a second portion comprising a plurality of arms, said arms comprising a first wire and a second wire, and providing a central body portion comprising a first wire and a second wire, wherein said first wire of said first portion, second portion, or central body portion is annealed at a first temperature and said second wire of said first portion, second portion or central portion is annealed at a second temperature. The first portion, second portion, and central body portion are assembled to form the flexible septal occluder.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a cutaway view of the heart illustrating an intracardiac defect.

FIG. 2 depicts a perspective view of an intracardiac occluder according to an illustrative embodiment of the invention.

FIG. 3 depicts a top plan view of a portion of an arm of a septal occluder made from a multi-strand bundle where the bundle is parted at a flexural point to create a gap in the strands according to an illustrative embodiment of the invention.

FIG. 4 depicts a top plan view of a portion of an arm of a septal occluder made from a multi-strand bundle where the bundle is parted at a flexural point to create a gap in the strands according to another illustrative embodiment of the invention.

FIG. 5 depicts a top plan view of a portion of an arm of a septal occluder made from a plurality of multi-strand bundles forming a cable where the cable is parted at a flexural point to create a gap in the strands according to another illustrative embodiment of the invention.

FIG. 6 depicts an exemplary cross-sectional view of the portion of the arm of the septal occluder of FIG. 5 according to an illustrative embodiment of the invention.

FIG. 7 depicts a top plan view of an exemplary occlusion shell of an exemplary intracardiac occluder with arms constructed of the multi-strand bundle shown in FIG. 3 according to another illustrative embodiment of the invention.

FIG. 8A depicts a top view of a portion of an arm of a septal occluder made from a multi-strand bundle forming a single loop, while FIG. 8B depicts a side view of the portion of the arm of FIG. 8A, according to an illustrative embodiment of the invention.

FIG. 9A depicts a top view of an occlusion shell of an illustrative intracardiac occluder with arms made from multi-strand bundles with loops as shown in FIG. 8A, while FIG. 9B depicts a side view of the exemplary occlusion shell and illustrative intracardiac occluder shown in FIG. 9A, where the arms of the occlusion shells are made from multi-strand bundles with loops as shown in FIG. 8A according to an illustrative embodiment of the invention.

FIG. 10A depicts a side view of a portion of an arm of a septal occluder made from a multi-strand bundle having multiple loops at the same point on the length of the arm forming a coil, while FIG. 10B depicts a top view of the portion of the arm shown in FIG. 10A, according to an illustrative embodiment of the invention.

FIG. 11A depicts a side view of a portion of an arm of a septal occluder made from a multi-strand bundle including loops of varying diameters along the length of the bundle, while

FIG. 11B depicts a top plan view of the portion of the arm of FIG. 11A, according to an illustrative embodiment of the invention.

FIG. 12A also depicts a side view of a portion of an arm of a septal occluder made from a multi-strand bundle comprising loops of varying diameters forming a coil along the length of the bundle, while FIG. 12B depicts a top plan view of the arm having loops forming a coil of FIG. 12A, according to another illustrative embodiment of the invention.

FIG. 13 depicts a portion of an arm of a septal occluder made from a multi-strand bundle, according to an illustrative embodiment of the invention.

FIGS. 14-17 depict various cross-sectional views of the strands that make up the arm of FIG. 13, according to illustrative embodiments of the invention. In FIG. 14, the cross-section of the strands is rectangular, while in FIGS. 15 and 17, the cross-section of the strands is triangular. In FIG. 16, the cross-section of the strands is hexagonal. The cross-sections depicted in FIGS. 14-17 are according to illustrative embodiments of the invention.

FIGS. 18-20 each depict a portion of an arm of a septal occluder made of a multi-stranded bundle, wherein the pitch of the component strands in each bundle differs from the pitch of the strands in the other exemplary bundles shown, according to illustrative embodiments of the invention.

FIGS. 21A-E depict multiple steps used to insert a septal occluder into a defect in a patient's heart according to an illustrative embodiment of the invention in a defect in a patient's heart.

### DETAILED DESCRIPTION OF THE INVENTION

The present invention is related to flexible intracardiac occluders, such as septal occluders, for the repair of intracardiac defects, such as, for example, a patent foramen ovale (PFO), an atrial septal defect, a ventricular septal defect, and left atrial appendages. All of the following embodiments of the invention include one or more features on the occluder to enhance flexibility at specific points on the occluder.

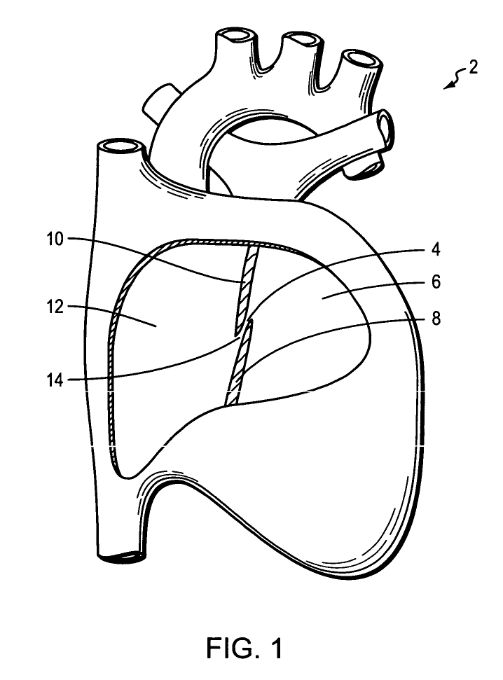

FIG. 1 depicts a cutaway view of a heart 2 illustrating an intracardiac defect 14. The heart 2 includes a septum 4 that divides the right atrium 12 from the left atrium 6. The septum 4 includes a septum primum 8, a septum secundum 10, and an exemplary intracardiac defect 14, which is to be corrected by the introduction of an intracardiac occluder of the present invention between the septum primum 8 and the septum secundum 10. Specifically, a PFO 14 is shown as an opening through the septum 4. The PFO 14 provides an undesirable communication between the right atrium 12 and the left atrium 6. Under certain conditions, a PFO 14 in the septum 4 can allow for the shunting of blood from the right atrium 12 to the left atrium 6. If the PFO 14 is not closed or obstructed in some manner, a patient is placed at high risk for an embolic stroke, migraine, or other physiological condition.

Septal Occluder

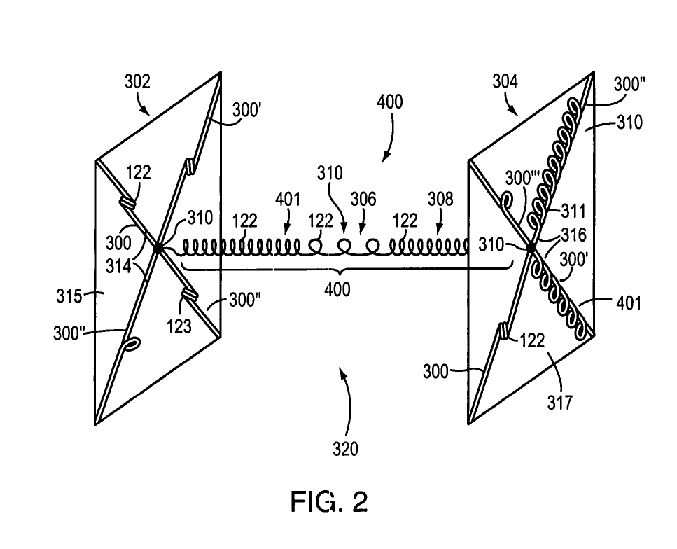

FIG. 2 depicts a perspective view of an intracardiac occluder 320 according to an illustrative embodiment of the invention. As shown, the intracardiac occluder 320 includes, for example, a proximal occlusion shell 302 (i.e., an occlusion shell that is closest to an operator of the intracardiac occluder 320 (e.g., a physician)), a distal occlusion shell 304, and a support structure 310. In one embodiment, the support structure 310 includes a proximal support structure 314 for supporting the proximal occlusion shell 302 and a distal support structure 316 for supporting the distal occlusion shell 304.

In one embodiment according to the invention, the support structures 314, 316 comprise a plurality of arms, generally, 300, for example, 300, 300′, 300″, 300′″. While the invention also contemplates an occlusion shell with no arms, the support structure 314, 316 may have one, two, three, four, five, six, seven, eight, nine, ten, or more arms supporting the occlusion shell 302, 304. Furthermore, while the invention contemplates an intracardiac occluder 320 with only one occlusion shell 302, 304, the invention also contemplates an intracardiac occluder 320 with two occlusion shells 302, 304, for example, a proximal occlusion shell 302 and a distal occlusion shell 304. In an embodiment where the intracardiac occluder 320 has two occlusion shells 302, 304, the proximal occlusion shell 302 has the same number of arms 300 as the distal occlusion shell 304, or alternatively, the proximal occlusion shell 302 has a different number of arms 300 than the distal occlusion shell 304.

With continued reference to FIG. 2, in one embodiment, the proximal occlusion shell 302 and the distal occlusion shell 304 are connected by a central body portion 400. In a further embodiment, the central body portion 400 joins the center of the proximal occlusion shell 302 to the center of the distal occlusion shell 304. In yet another embodiment, the occlusion shells 302, 304 rotate about the axis of the central body portion 400. While the occlusion shells 302, 304 depicted in FIG. 2 are rectangular, the shells may be circular, elliptical, square, convex, concave, flat, or be any other functional shape.

As shown in FIG. 2, according to the invention, in one embodiment the occlusion shells 302, 304 include a scaffold 315, 317 supported by the proximal and distal support structures 314, 316. In one embodiment, the scaffold 315, 317 is made from a biological tissue, such as collagen. For example, in one embodiment, the scaffold 315, 317 comprises collagen derived from the tunica mucosa layer of the porcine small intestine, or from other sources as described in, for example, U.S. Patent Application Publication No. 2004-0098042, incorporated by reference herein. Alternatively, the scaffold 315, 317 of occlusion shells 302, 304 is a synthetic scaffold, such as a polyester fabric, expanded polytetrafluoroethylene (ePTFE), polyvinyl alcohol (e.g. Ivalon®), a metal mesh, or a bioresorbable material. In a further embodiment, the scaffold 315 of the proximal occlusion shell 302 is the same material as the scaffold 317 of the distal occlusion shell 304, while in another embodiment, the scaffold 315 of the proximal occlusion shell 302 is a different material than the scaffold of the distal occlusion shell 304.

Multi-Strand Bundles

A key aspect of the invention is that intracardiac occluders made with single strands of wire or with multi-strand bundles modified according to the invention have improved mechanical properties. Wire strands of the invention may be made from a suitable metal, such as stainless steel, nitinol, or MP35N, or they may be made from a polymer or a bioresorbable material. According to the invention, single strands alone, or multi-strand bundles can be modified to provide beneficial results, such as, improving the flexibility of the intracardiac occluder 320, enhancing the conformability of the occluder 320 to the intracardiac defect 14, and enhancing the apposition of the occlusion shells 302, 304 to one another and the intracardiac defect 14, thereby reducing trauma to intracardiac tissues, providing faster rates of tissue ingrowth and hastening defect closure rates. The multi-strand bundles of the invention may be used create the support structure 310 of the intracardiac occluder 320, including the arms 300 and/or the central body portion 400.

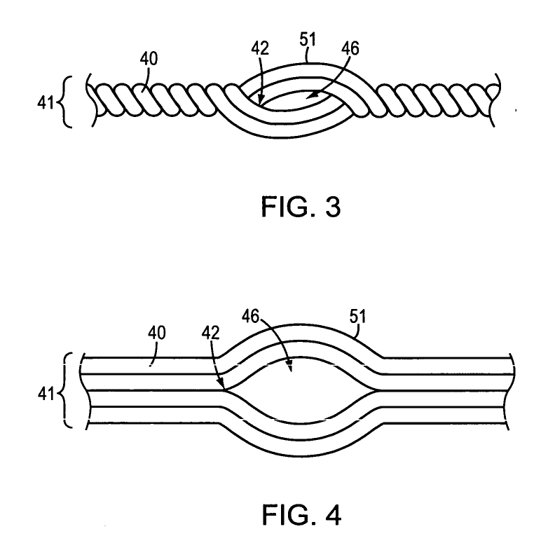

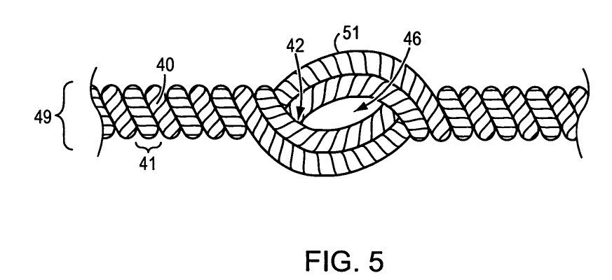

FIGS. 3 and 4 depict top plan views of a portion of an arm 300 of a septal occluder 320 made from a multi-strand bundle 41 where the bundle 41 is parted at a flexural point 51 to create a gap 46 in the strands 40 according to an illustrative embodiment of the invention. Multi-strand bundles 41 are created by weaving, braiding, twisting, bundling, winding, or otherwise grouping a plurality of individual wire strands 40 to form a bundle 41 of strands 40 of the arm 300 of the septal occluder 320. For example, FIG. 3 depicts an exemplary multi-strand bundle 41 of a portion of an arm 300 according to an illustrative embodiment of the invention in which the strands 40 are twisted together. For example, FIG. 4 depicts a top plan view of an exemplary multi-strand bundle 41 according to another illustrative embodiment of the invention in which an arm 300 of a septal occluder 320 is made from a multi-strand bundle 41 in which the strands 40 are bundled to run parallel to their lengthwise axis. In a further embodiment, the strands 40 are bundled to run parallel to the lengthwise axis of the arm 30. As illustrated in FIGS. 3 and 4, the arm 300 may include four strands 40 of wire to form a bundle 41. Alternatively, the arm 300 may include, for example, two, three, five, six, seven, eight, nine, ten or more wire strands 40.

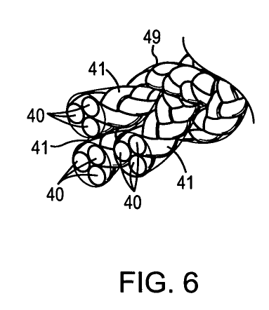

FIG. 5 depicts a top plan view of a portion of an arm 300 of a septal occluder 320 made from a plurality of multi-strand bundles 41 forming a cable 49 where the cable 49 is parted at a flexural point 51 to create a gap 46 in the strands 40, according to an illustrative embodiment of the invention. As shown in FIG. 5, an exemplary plurality of multi-strand bundles 41 may be grouped together to form a cable 49 to be used in one or more arms 300. The plurality of multi-strand bundles 41 forming the cable 49 may also be used to make the central body portion 400 (not shown).

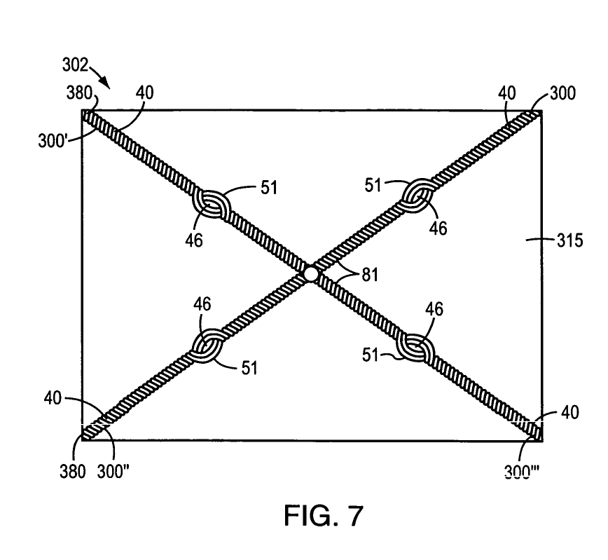

FIG. 6 depicts an exemplary cross-sectional view of the portion of the arm 300 of the septal occluder 320 illustrated in FIG. 5. As shown in FIG. 6, three multi-strand bundles 41, each including three strands 40, are bundled together to create a cable 49. The number of bundles in the cable 49 and the number of strands 40 in each of the multi-strand bundles 41 is not limited to what is illustrated in FIG. 6. For example, the cable 49 may comprise two, three, four, five or more multi-strand bundles and each of the multi-strand bundles may comprise two, three, four, five or more strands 40 (not shown). Furthermore, each of the multi-strand bundles 41 in the cable 49 need not be the same. For example, in one embodiment, one multi-strand bundle 41 is made of three strands 40, while another multi-strand bundle is made of seven strands 40 (not shown). Additionally, the multi-strand bundles 41 or cables 49 can be used to form the central body portion 400 of an occlusion shell support structure 310 of a septal occluder 320.

Modifications of the Multi-Strand Bundles

The invention contemplates modifying the multi-strand bundles 41 which form an arm or arms 300 and/or the central body portion 400 of an intracardiac occluder in order to improve articulation of the intracardiac occluder 320 in the intracardiac defect. Enhanced conformability, increased flexibility and reduced bending stiffness of the occlusion shells 302, 304 allows the septal occluder 320 according to the invention to conform to the tissue contacted by the occluder 320, thereby reducing trauma to the tissue, and increasing the defect closure rate. In order to accomplish these objectives, the multi-strand bundles 41 may be modified according to illustrative embodiments of the invention as described below.

Referring again to FIG. 3 and FIG. 4, in one embodiment according to the invention, a flexural point 51 can be achieved by introducing a part 42 amongst the strands 40 by separating one or more strands 40 from the other strands 40 in the multi-strand bundle 41 to form a gap 46. For example, at least one strand 40 is parted from the remaining strand or strands 40 to form the gap 46, the gap 46 being defined by the parted strands. In an alternative embodiment, as shown in FIG. 5, a flexural point 51 in the form of gap 46 may also be achieved by forming the gap 46 by creating a part 42 between the multi-strand bundles 41 forming the cable 49. For example, at least one multi-strand bundle 41 is parted from the remaining multi-strand bundle or bundles 41 to form the gap 46.

According to one embodiment of the invention, a flexural point 51 is disposed on at least one arm 300 of an occlusion shell 302, 304. In another embodiment, a flexural point 51 may be disposed on more than one arm 300 of an occlusion shell 302, 304. The invention further contemplates an arm 300 with more than one flexural point 51. For example, an arm 300 may have one, two, three, four, five or more flexural points 51. In one embodiment, a flexural point 51 occurs near or at the tip 380 of the arm 300, while in another embodiment, a flexural point does not occur at the tip 380 of the arm 300. In another embodiment, the flexural point 51 may occur anywhere along the length of the arm 300. In one embodiment, the flexural point 51 is a gap 46.

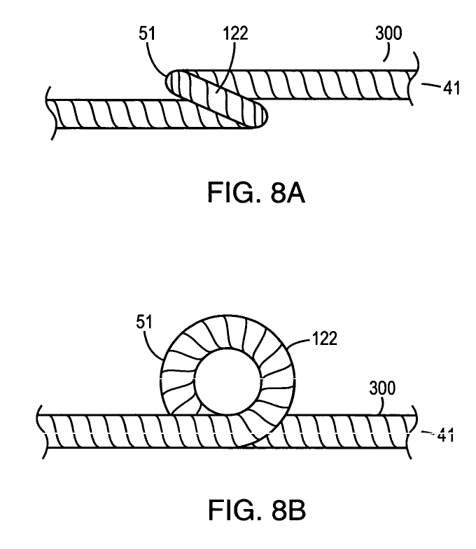

FIG. 7 depicts a top view of an exemplary occlusion shell 302 of an exemplary intracardiac occluder 320 with arms 300 constructed of the multi-strand bundle shown in FIG. 3, according to an illustrative embodiment of the invention. According to the illustrative embodiment, the occlusion shell 302, 304, for example the proximal occlusion shell 302, has four arms 300, 300′, 300″, 300′″. In one embodiment, at least one arm 300 is made of a multi-strand bundle 41 with the at least one arm 300 having a gap 46 between the strands 40 of the multi-strand bundle 41 forming a flexural point 51. In another embodiment, all arms 300 of the occlusion shell 302, 304 are made of multi-strand bundles 41, with each arm having a gap 46 between the strands 40 of the multi-strand bundle 41 forming a flexural point 51. The flexural point 51 enhances flexibility of the arm 300 by decreasing the arm's 300 resistance. The flexural point 51 also increases the arm's 300 surface area, which reduces trauma to the surrounding tissue and improves stability of the septal occluder 320 in the defect. These features improve the ability of the septal occluder 320 to seal the defect in which the septal occluder 320 is inserted.

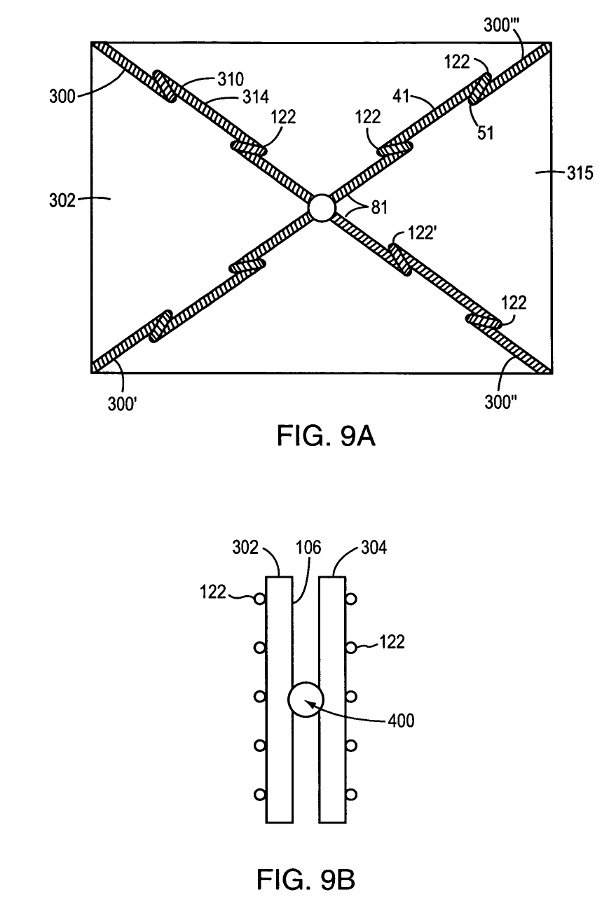

According to another embodiment of the invention, flexural points 51 may also be created through the use of loops, generally, 122. For example, FIG. 8A depicts a top view of a portion of an arm 300 of a septal occluder 320 made from a multi-strand bundle 41 forming a single loop 122 according to the invention. FIG. 8B depicts a side view of the single loop 122 shown in FIG. 8A. The single loop 122 forms a flexural point 51 along the length of the arm 300.

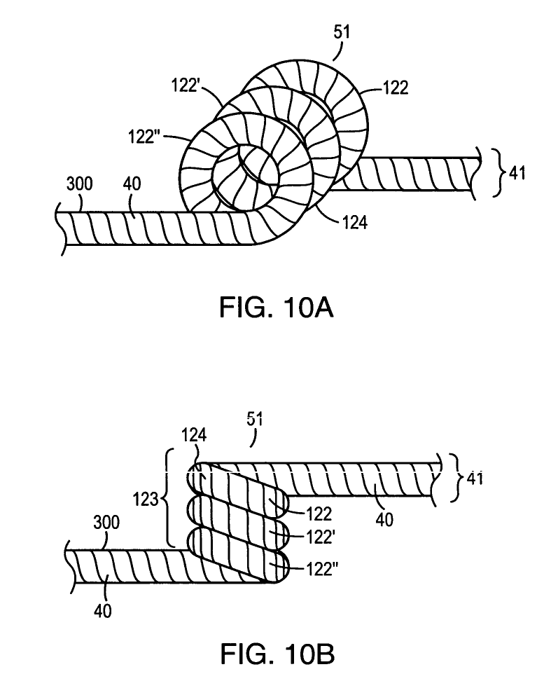

FIG. 9A depicts a top view of an occlusion shell 302 of an illustrative intracardiac occluder 320 with arms 300 made from multi-strand bundles 41 with loops 122 as shown in FIG. 8A, while FIG. 9B depicts a side view of the view of the exemplary occlusion shell 302 and illustrative intracardiac occluder 320 shown in FIG. 9A, where the arms 300 of the occlusion shell 302, 304 are made from multi-strand bundles 41 with loops 122 as shown in FIG. 8A, according to an illustrative embodiment of the invention. As shown in FIG. 9A, the support structure 310, for example, the proximal support structure 314, may have one or more arms 300 as previously discussed. In one embodiment, at least one arm 300 of the support structure 314 has a loop 122, whereas in another embodiment, more than one arm 300 of the support structure 314 has a loop 122. In another embodiment, at least one arm 300 has more than one loop 122. As shown in FIGS. 9A and 9B, when more than one loop 122 is present on one arm 300, in one embodiment the loops 122 are spaced along the length of the arm 300, thus creating a flexural point 51 at each point on the arm 300 where a loop 122 is present. In one embodiment, each loop 122 has the same diameter as each other loop 122 on the arm 120, whereas in another embodiment, at least one loop 122 on an arm 300 has a different diameter than a second loop 122 on the arm 300 as discussed below.

Flexural points 51 may also be created through the use of a coil 123, according to an illustrative embodiment of the invention. In one embodiment, a coil 123 as used herein is defined as two or more consecutive loops 122 on the strand 40 that forms the arm 300 of an occlusion shell. Alternatively, in another embodiment, a coil 123 as used herein defines two or more consecutive loops on a multi-strand bundle 41 making up a cable 49. For example, a coil 123 can include two, three, four, five or more loops 122.

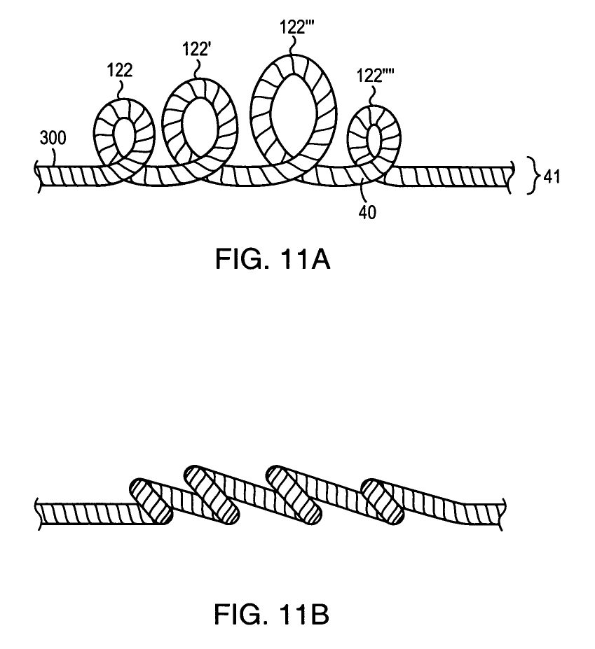

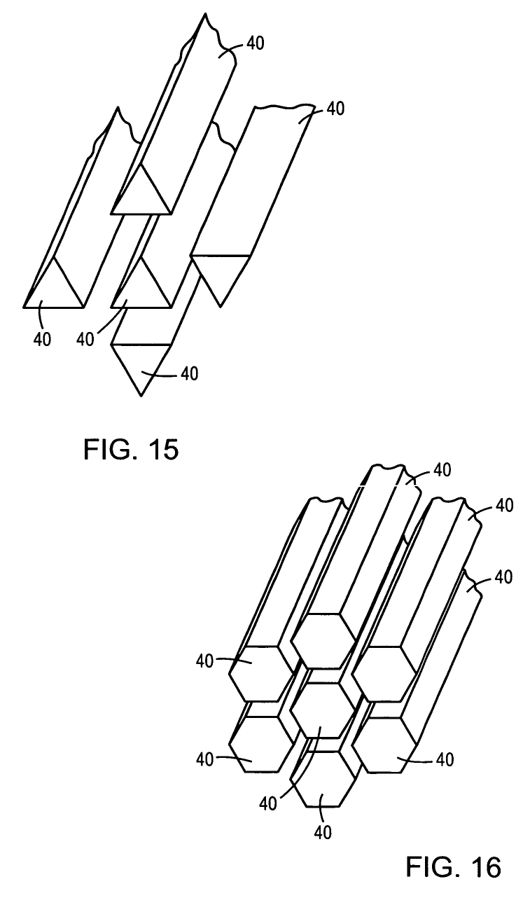

FIG. 10A depicts a side view of a portion of an arm of a septal occluder 320 made from a multi-strand bundle 41 having multiple consecutive loops 122 at the same point on the length of the arm 300 forming a coil 123, while FIG. 10B depicts a top view of the portion of the arm 300 shown in FIG. 10A, according to an illustrative embodiment of the invention. In one embodiment, multiple consecutive loops 122, 122′, 122″ forming a coil 123 are present at one flexural point 51 on an arm 300. In one embodiment, the loops 122 forming the coil 123 are parallel or substantially parallel to each other, while being perpendicular to the length of the arm 300, while in another embodiment, the loops 122 forming the coil 123 are parallel or substantially parallel to each other, and are also parallel or substantially parallel to the length of the arm 300. In another embodiment, the loops 122 present at the same flexural point 51 may be of the same diameter as shown in FIG. 10A, or they may be of different diameters as discussed below in relation to FIGS. 12A and B.

With continued reference to FIGS. 10A and B, in a further embodiment, an arm 300 has more than one flexural point 51 with multiple consecutive loops 122, 122′, 122″ forming a coil 123 at each flexural point 51 along the length of the arm 300. In yet another embodiment, an arm has multiple flexural points 51, where at least one flexural point has a single loop 122, as shown, for example, in FIG. 8A, and at least one other flexural point 51 has multiple loops 122, 122′, 122″ forming a coil 123, as shown, for example, in FIG. 10A. In another embodiment, the central body portion 400, which joins at least two occlusion shells 302, 304, may also have multiple loops 122, 122′, 122″ present at one flexural point 51 on the central body portion 400 forming a coil 123. In another embodiment, each loop 122 forming a coil 123 at one flexural point 51 may be of the same or different diameter, as discussed below in relation to FIGS. 12A and B.

Referring again to FIG. 2, in another embodiment, a single strand or multi-strand bundle includes a helical coil 401. The helical coil 401 is formed by rotating the strand or bundle in a constantly changing plane around a central axis and includes multiple loops 122, with each loop 122 being at a different point along the central body portion 400. For example, in one embodiment, the helical coil 401 has two, three, four, five, six, seven, eight, nine, ten or more loops 122. In one embodiment, according to the invention, the helical coil 401 can be used to connect two occlusion shells 302, 304 as shown in FIG. 2. Additionally, all or a portion of the length of the central body portion 400 includes the helical coil 401. The helical coil 401, for example, is a compression coil or alternatively, a tension coil.

According to yet another embodiment of the invention, as shown in FIG. 2, one or more arms 300 include a helical coil 401 for at least a portion of the length of the arm 300. The helical coil 401 is formed by rotating the strand or bundle in a constantly changing plane around a central axis and includes multiple loops 122, with each loop 122 being at a different point along the arm 300. For example, in one embodiment, each loop 122 of the helical coil 401 creates a flexural point 51 along the length of the arm 300. In one embodiment the helical coil 401 along the length of the arm 401 is a compression coil, or alternatively, a tension coil.

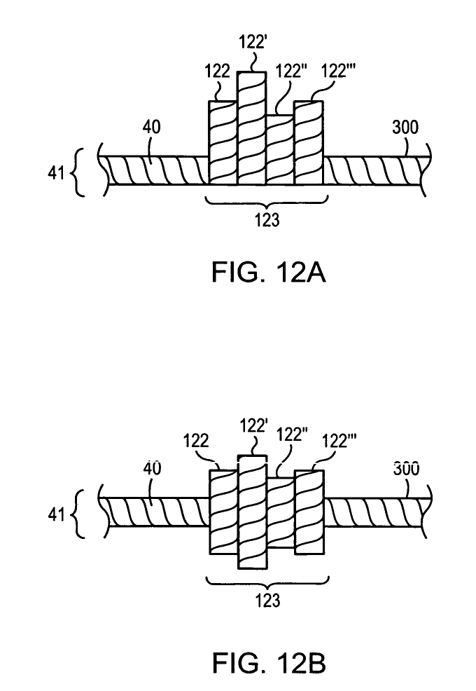

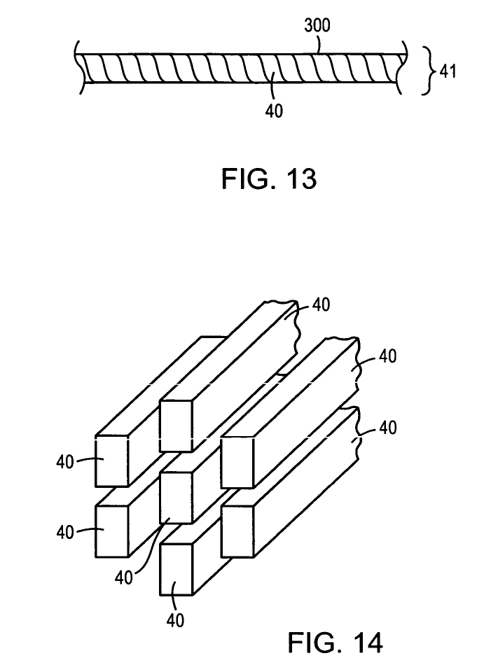

FIG. 11A depicts a side view of a portion of an arm 300 of a septal occluder 320 made from a multi-strand bundle 41 including loops 122 of varying diameters along the length of the bundle 41 forming the arm 300, while FIG. 11B depicts a top plan view of the portion of the arm shown in FIG. 11A, according to an illustrative embodiment of the invention. In one embodiment, when the arm 300 includes multiple loops 122, for example, loops 122, 122′, 122″, the diameter of the loops 122 can vary.

Alternatively, referring again to FIG. 2, the central body portion 400 may also include loops 122 of varying diameter along the length of the central body portion 400. For example, FIG. 2 depicts an embodiment wherein the central body portion 400 includes loops 122 of varying diameter, i.e, loops 122 of a smaller diameter in region 306 are positioned between loops 122 of a larger diameter in region 308. Loops 122 varying in diameter may be present on an individual strand 40, on a multi-strand bundle 41, or on a cable 49 of the central body portion 400, and/or on one or more arms 300 of an occlusion shell, for example, the proximal occlusion shell 302. Also, loops 122 varying in diameter may be present at the same flexural point 51 on the arm 300 or the central body portion 400. The variation in diameter of the loops 122 enhances the flexibility of the intracardiac occluder, including the arms 300 and the central body portion 400.

In addition, when a loop or loops 122 are present on an arm 300 or on the central body portion 400, a safety wire (not shown) can be attached to the arm 300 or the central body portion 400 to prevent overextension of the loop or loops 122. In one embodiment, the safety wire runs through the inner lumen of a coil 123 and is attached to the first loop and last loop 122 of a coil 123 positioned on an arm 300 or central body portion 400. Alternatively, the safety wire is attached to the first loop and last loop 122 of a coil 123, and runs along the outer surface 124 of the coil 123. The safety wire may be made from a suitable metal, such as stainless steel, nitinol, or MP35N, or it may be made from a polymer or a bioresorbable material.

FIG. 12A depicts a side view of a portion of an arm 300 of a septal occluder 320 made from a multi-strand bundle 41 comprising a plurality of loops 122 of varying diameters forming a coil 123 along the length of the bundle 41, while FIG. 12B depicts a top plan view of the arm 300 having loops 122 forming a coil 123 as illustrated in FIG. 12A. According to this illustrative embodiment of the invention, the loops 122 are parallel to each other while being perpendicular or substantially perpendicular to the length of the arm 300 or central body portion 400 on which the loops 122 are disposed. The loops 122, 122′, 122″, and 122′″ in this embodiment all vary in diameter compared to one another. In yet another embodiment, at least two loops 122 of differing diameters are parallel to each other, but are perpendicular or substantially perpendicular to the length of the arm 300 or central body portion 400 on which the loops 122 are disposed. In yet another embodiment, at least two loops 122 of differing diameters are parallel to each other, and are parallel or substantially parallel to the length of the arm 300 or the central body portion 400 on which the loops 122 are disposed.

According to a further embodiment of the invention, each loop 122 need not be of a different diameter than another loop 122. For example, an arm 300 or central body portion 400 may have two or more loops 122 of one diameter, as well as a loop or loops 122 of a second differing diameter. According to one embodiment of the invention, at least two loops 122 of the same diameter are parallel to each other, but are perpendicular or substantially perpendicular to the length of the arm 300 or central body portion 400 on which the loops 122 are disposed. In a further embodiment, a safety wire runs through the inner lumen of the loops 122 of a coil 123 and is attached to the first and last loop 122 of the coil 123 positioned on an arm 300 or central body portion 400. Alternatively, the safety wire is attached to the first and last loop 122 of a coil 123, and runs along the outer surface 124 of the coil 123. The safety wire may be made from a suitable metal, such as stainless steel, nitinol, or MP35N, or it may be made from a polymer or a bioresorbable material.

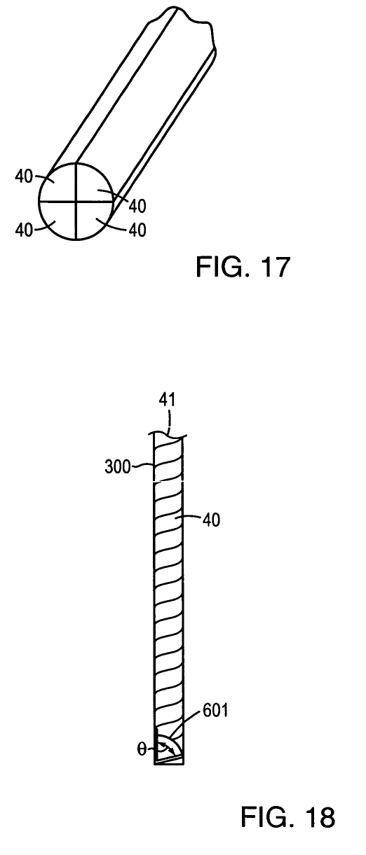

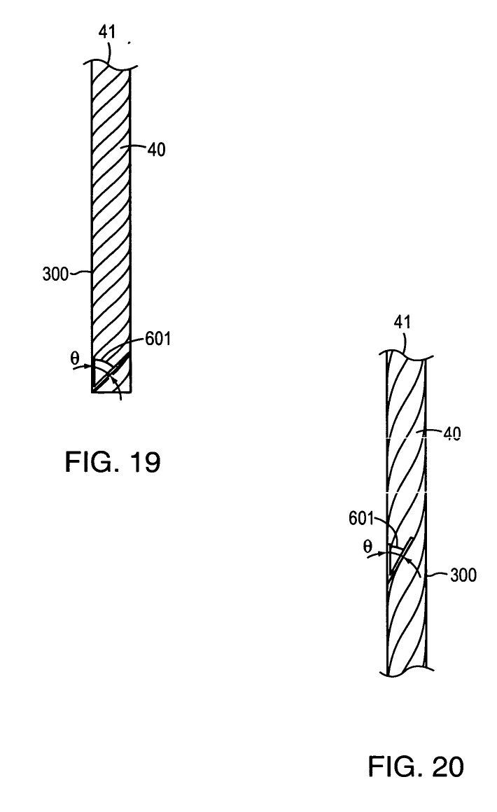

According to the invention, varied flexibility and conformability of a septal occluder can also be achieved by varying the shape of the strands in a multi-strand bundle. FIG. 13 depicts a portion of an arm 300 of a septal occluder made from a multi-strand bundle 41 according to an illustrative embodiment of the invention, while FIGS. 14-17 depict various cross-sectional views of the strands 40 that make up the arm 300 depicted in FIG. 13, according to illustrative embodiments of the invention. The strands 40 forming the multi-strand bundles 41 of the arms 300 may include a non-circular cross-section. For example, as illustrated in FIGS. 15 and 17, the cross-section of the strands 40 is triangular. A triangular strand cross-section produces a higher point pressure along the edge of the strand formed by the apices of the triangle. Conversely, the flat side of a triangular strand may aid in minimizing trauma to the cardiac tissues by eliminating pressure points. In another embodiment, the strands 40 may have a rectangular or ribbon-like cross-section as shown in FIG. 14, while in yet another embodiment, shown in FIG. 16, the cross-section of the strands 40 is hexagonal. Additionally, the cross section of a strand 40 may be circular, or alternatively, any non-circular geometric shape. For example, an elliptical, rectangular, rhomboidal, trapezoidal, or any other polygonal or non-circular geometric shaped cross-section may be used.

Furthermore, in one embodiment, at least one strand 40 of the multi-strand bundle 41 forming an arm 300 of an septal occluder 320 comprises a circular cross-section. For instance, in one embodiment, the cross-section of one or more strands 40 is circular. In another embodiment (not shown), the cross-section of one strand 40 is circular while the cross-section of one or more strands 40 is triangular. Alternatively, the invention also features mixing two or more non-circular strands 40 to form multi-strand bundles 41 (not shown). For example, in one embodiment (not shown), the cross-section of one or more strands 40 is circular, while the cross-section of one or more strands 40 is triangular, and the cross-section of one or more strands 40 is octagonal. Optionally, in another embodiment, the cross-section of one or more strands 40 is square, while the cross-section of one or more strands 40 is triangular, and the cross-section of one or more strands 40 is pentagonal.

With continued references to FIGS. 13-17, the invention contemplates that the support structure 310 of an intracardiac occluder 320, such as an arm or arms 300 and/or the central body portion 400 is comprised of any of the embodiments of multi-strand bundles of non-circular or circular cross-section as described above. For example, in one embodiment, an occlusion shell 312, 314 has at least one arm 300 comprising a strand with a non-circular cross-section. An occlusion shell 312, 314 may have two or more arms 300 comprising a strand of non-circular cross-section.

According to the invention, varied flexibility and conformability of a septal occluder can also be achieved by varying the diameter or thickness of the strands 40 in a multi-strand bundle 41. For example, in one embodiment (not shown), the diameter of at least one strand 40 is larger than the diameter of another strand 40, or in other words, at least one strand 40 is thicker than another strand 40. There may be as many diameters or thicknesses of strands 40 as there are strands 40 in a bundle 41. Multi-strand bundles 41 made from strands 40 of varying thicknesses or diameters may be used to create an arm or arms 300 and/or the central body portion 400 of an intracardiac occluder 320.

According to the invention, varied flexibility and conformability of a septal occluder can also be achieved by varying the length of strands 40 in a multi-strand bundle 41. For example, in one embodiment (not shown), at least one strand 40 has a greater length than another strand 40 in a bundle 41. However, there may be one, two, three, four, five, six or more lengths represented in a multi-strand bundle 41 of strands 40. It is possible to have as many representative lengths of strands 40 as there are strands 40 in that multi-strand bundle 41. For example, in one embodiment, a strand has a first length, another strand has a second length, and yet another strand has a third length. In yet another embodiment, each strand 40 differs in length from every other strand in the multi-strand bundle 41. While the length of strands 40 in a bundle 41 may differ, the bundle 41 may also include strands 40 of the same length. For example, in one embodiment, at least one strand 40 differs in length from at least one other strand 40, and at least one strand 40 is equal in length to at least one other strand 40. Multi-strand bundles 41 of strands 40 may be used to create an arm or arms 300 and/or the central body portion of an intracardiac occluder 320.

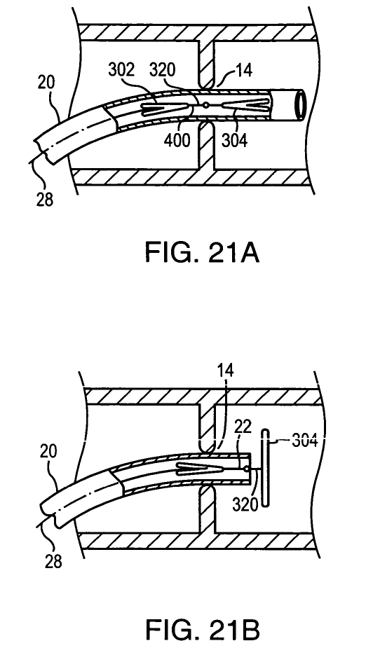

Varied flexibility and conformability of a septal occluder can also be achieved by varying the pitch of the strands in a multi-strand bundle. FIGS. 18-20 each depict a portion of an arm 300 of a septal occluder 320 made of a multi-strand bundle 41, wherein the pitch of the component strands 40 in each multi-strand bundle 41 differs from the pitch of the strands 40 in the other exemplary multi-strand bundles 41 shown, according to illustrative embodiments of the invention. According to the illustrative embodiments, the pitch of the strands 40 in a multi-strand bundle 41, or the pitch of the multi-strand bundle 41 in a cable 49 may be altered to increase or decrease its flexibility. As the angle θ 601 defining the pitch of the strands 40 in a multi-strand bundle 41, or the pitch of the multi-strand bundle in a cable 49 increases toward 90°, the bending stiffness of the bundle 41 or cable 49 decreases and the flexibility increases. As the angle θ 601 defining the pitch of the strands 40 in a multi-strand bundle 41, or the pitch of the multi-strand bundle in a cable 49 decreases toward 0°, the bending stiffness of the bundle 41 or cable 49 increases and the flexibility decreases.

Multi-strand bundles 41 or portions of multi-strand bundles 41 annealed at various temperatures may also be used in the arm or arms 300 and/or the central body portion 400 of the intracardiac occluder 320 according to the invention in order to affect the flexibility and conformability of the septal occluder. For example, in a multi-strand bundle 41, at least one strand 40 is annealed at a different temperature than at least one other strand 40 in the multi-strand bundle 41. In a further embodiment, one or more strands 40 in a multi-strand bundle 41 is annealed at a different temperature than at least one other strand 40 in the bundle 41. In a further embodiment, a bundle 41 may have one or more strands 40 subjected to a first annealing temperature, one or more strands 40 subjected to a second annealing temperature, and one or more strands 40 subjected to a third annealing temperature. In a further embodiment, each strand 40 has a different annealing temperature than every other strand 40 in the bundle 41. For example, there may be as many representative annealing temperatures as there are strands 40 in a given multi-strand bundle 41.

The advantage of varying the annealing temperatures of the various strands 40 is that different temperatures impart different mechanical properties to the multi-strand bundle 41. For example, in one embodiment stiffer strands 40 are positioned in the core of a multi-strand bundle and more pliable strands 40 are positioned on the outer segments of a multi-strand bundle. In another embodiment, it is beneficial to interweave softer strands 40 together, while in another embodiment, weaving stiffer strands 40 together is advantageous. For example, in one embodiment, the strands 40 of the multi-strand bundle 41 are made of nitinol, with at least one strand 40 being heat treated to impart enhanced flexibility to the strand 40, while at least one other strand 40 is heat treated to impart greater stiffness to that strand 40.

In another embodiment according to the invention, strands that form arms 300 of the septal occluder 320 may be subjected to one or more annealing temperatures along their lengths.

For example, a strand 40 is annealed at a higher temperature at the center, but annealed at a lower temperature towards the ends of the strand. Multi-strand bundles 41 made from strands annealed at different temperatures at different points along the strands' lengths produces multi-strand bundles 41 that have more pliable regions along the lengthwise axis of the multi-strand bundle 41 intermixed with stiffer regions along the length of the multi-strand bundle 41. This aids in reducing trauma by permitting flexion of the multi-strand bundles 41 at the more pliable regions along the length of the bundles 41. In one embodiment of the invention, an occlusion shell 302 of an intracardiac occluder 320 comprises an arm 300 comprising a multi-strand bundle 41 where at least one strand 40 in the arm 300 is annealed at a different temperature than at least one other strand 40 in the same arm 300. In a further embodiment, an occlusion shell 302 of an intracardiac occluder 320 comprises one or more arms 300 comprising a multi-strand wire bundle 41 and in each arm 300, at least one strand 40 in the arm 300 was annealed at a different temperature than at least one other strand 40 in the same arm 300. In yet another embodiment, an occlusion shell 302 comprises one or more arms 300 comprising a multi-strand bundle 41 wherein at least one strand 40 in the multi-strand bundle has been subjected to two or more annealing temperatures along its length. In another embodiment of the invention, the central body portion 400 can comprise a multi-strand bundle 41 where at least one strand 40 in the central body portion 400 was annealed at a different temperature than at least one other strand 40 in the central body portion 400. In yet another embodiment, an arm or arms 300 and/or the central body portion 400 of an intracardiac occluder 320 may comprise any of the multi-strand bundles with strands 40 annealed at temperatures as described above.

While many of the modifications discussed above have been discussed in the context of the arms 300 of an occlusion shell support structure 314, these variations are equally applicable to any individual strand 40, multi-strand bundle 41, or cable 49 comprising the central body portion 400 of the intracardiac occluder 320 according to the invention. Furthermore, multi-strand bundles 41 of the invention may comprise multiple modifications within the same bundle. For example, a multi-strand bundle 41 forming an arm or arms 300 and/or the central body portion 400 of a flexible intracardiac occluder 320 may include any one or more of the following modifications including, but not limited to varying the number of strands 40 in a multi-strand bundle 41, varying the number of multi-strand bundles 41 in a cable 49, and varying the cross-sectional geometry of the individual strands 40, varying the diameter or thickness of individual strands 40, varying the length of individual strands 40, varying the annealing temperature of individual strands 40, varying the pitch of the strands 40 in a multi-strand bundle 41, varying the diameter of loops 122, and adding gaps 46 or loops 122, including coils 123 or helical coils 401 to a multi-strand bundle.

Deployment of a Intracardiac Occluder

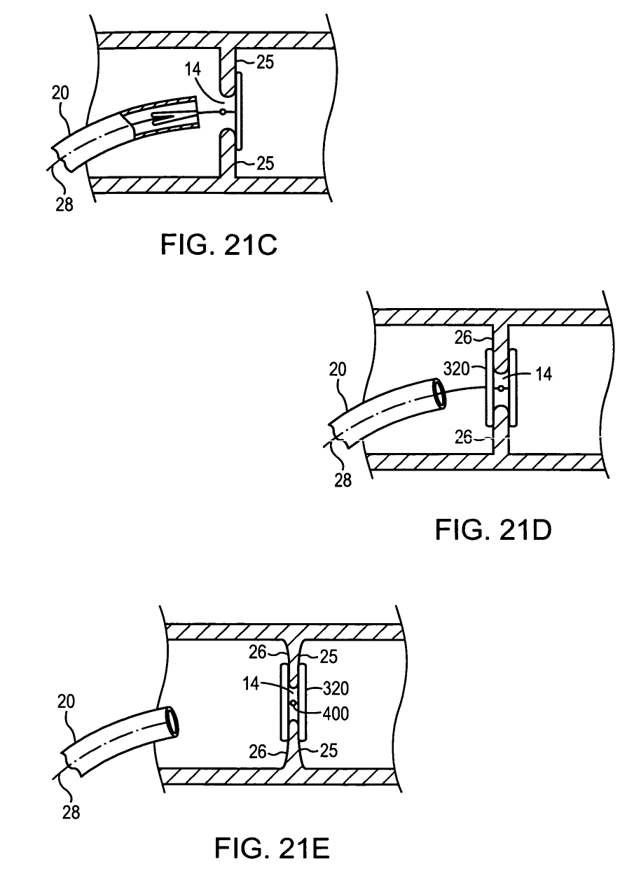

FIGS. 21A-21E depict multiple steps used to insert a septal occluder in a defect in a patient's heart according to an illustrative embodiment of the invention. The intracardiac occluder 320 according to the invention described above is delivered percutaneously and transvascularly via a catheter 20 and guided to a predetermined location, for example, to a PFO 14.

As shown in FIG. 21A, in one embodiment of the invention, a collapsed septal occluder 320 is inserted into a catheter 20 with the distal occlusion shell 304 collapsed and positioned distally to the proximal occlusion shell 302. The catheter 20, with the collapsed intracardiac occluder 320 contained in a distal portion thereof is inserted into a blood vessel of a patient and is navigated through the patient's blood vessels into the heart and, for example, crosses the intracardiac defect 14.

As shown in FIG. 21B, according to one embodiment of the invention, the intracardiac occluder 320 is deployed beyond the distal end of the catheter 20 to cause the distal occlusion shell 304 to exit the distal end of the catheter 20. Such deployment can be accomplished either by advancing the intracardiac occluder 320 within the catheter by means of, for example, advancing a positioning wire 28 joined to the intracardiac occluder 320 distally while holding the catheter 20 in place, or by retracting the catheter 20 while proximally holding the intracardiac occluder 320 in place with the positioning wire 28. Once the distal occlusion shell 304 has been advanced beyond the distal end of the catheter 20, the occlusion shell 304 will automatically and resiliently open to its expanded configuration.

As shown in FIG. 21C, the catheter 20 and intracardiac occluder 320, in one embodiment, are then retracted to seat the distal occlusion shell 304 against the distal wall 25 of the defect 14 to occlude the defect 14. As shown in FIG. 21D, in one embodiment, the catheter sheath 20 is further withdrawn proximally to allow the proximal occlusion shell 302 to be deployed from the distal end of the catheter 20. Once deployed, the proximal occlusion shell 302 opens automatically and resiliently in same manner as the distal occlusion shell 304.

As shown in FIG. 21E, upon deployment, the proximal occlusion shell 302 lies against the proximal wall 26 of the intracardiac defect 14 thereby occlusion the defect 14 on the proximal side. The catheter 20 and position wire 28 are then withdrawn from the patient leaving the deployed intracardiac occluder 320 with the occlusion shells 302, 304 positioned on each side of the intracardiac defect 14. Because the occlusion shells 302, 304 are free to move relative to each other, being able to rotate about the axis of the central body portion 400, the intracardiac occluder 320 can be used in applications in which it is desirable that the occluder elements are not directly opposed to one another. For example, such an intracardiac occluder 320 can be used to correct flap-like or tunnel-like defects in the atrial septum, other intracardiac defects.

Variations, modifications, and other implementations of what is described herein will occur to those of ordinary skill in the art without departing from the spirit and the scope of the invention as claimed. Accordingly, the invention is to be defined not by the preceding illustrative description, but instead by the spirit and scope of the following claims.

What is claimed is:

1. A flexible septal occluder, comprising:
a first portion comprising a plurality of arms and a flexural point disposed on at least one of said arms;
a second portion comprising a plurality of arms; and
a central body portion joining said first portion with said second portion,
wherein at least one of said arms comprises a plurality of strands, said plurality of strands forming a bundle of strands interrupted at said flexural point by a gap in said strands.

2. The flexible septal occluder of claim 1, wherein more than one flexural point is disposed on at least one of said arms of said first portion.

3. The flexible septal occluder of claim 1, wherein at least one flexural point is disposed on each arm of said first portion.

4. The flexible septal occluder of claim 1, further comprising at least one flexural point disposed on said central body portion.

5. The flexible septal occluder of claim 1, wherein said plurality of strands runs parallel to one another along the lengthwise axis of the arm.

6. The flexible septal occluder of claim 1, wherein said plurality of strands runs parallel to one another along the lengthwise axis of the central body portion.

7. The flexible septal occluder of claim 1, wherein at least one flexural point is disposed on at least one arm of said second portion.

8. A flexible septal occluder, comprising:
a first portion comprising a plurality of arms, at least one of said arms comprising a plurality of strands forming a bundle of strands, said bundle of strands comprising a first loop and a second loop; and
a second portion comprising a plurality of arms; and
a central body portion joining said first portion with said second portion,
wherein said first loop comprises a different diameter than said second loop, thereby enhancing flexibility of said septal occluder.

9. The flexible septal occluder of claim 8, wherein the diameter of said first loop is the same diameter as the diameter of another loop disposed on said first portion.

10. The flexible septal occluder of claim 9, wherein said first loop and said another loop are adjacent.

11. The flexible septal occluder of clam 8, wherein said first loop and said second loop are adjacent.

12. The flexible septal occluder of claim 8, wherein said second portion comprising a plurality of arms further comprises at a plurality of strands forming a bundle of strands, said bundle of strands comprising a first loop and a second loop.

13. The flexible septal occluder of claim 8, wherein at least one of said plurality of arms of said first portion or said second portion comprises a coil.

14. The flexible septal occluder of claim 8, wherein said central body portion comprises a coil.

15. A flexible septal occluder, comprising:
a first portion comprising a plurality of arms;
a second portion comprising a plurality of arms; and
a central body portion joining said first portion with said second portion, said central body portion comprising a plurality of strands forming a bundle of strands, a first loop and a second loop, wherein said first loop comprises a different diameter than said second loop, thereby enhancing flexibility of the septal occluder.

16. The flexible septal occluder of claim 15, wherein the first loop and the second loop are adjacent.

17. The flexible septal occluder of claim 15, wherein the central body portion comprises another loop with the same diameter as the first loop.

18. The flexible septal occluder of claim 17, wherein the first loop and said another loop are adjacent.

19. A flexible septal occluder, comprising:
a first portion comprising a plurality of arms comprising a plurality of strands;
a second portion comprising a plurality of arms comprising a plurality of strands; and
a central body portion comprising a plurality of strands, wherein one of the strands of the first, second, or central body portions comprises a non-circular cross-section.

20. The flexible septal occluder of claim 19 wherein said non-circular cross-section is a triangular cross-section.

21. The flexible septal occluder of claim 19 wherein said non-circular cross-section is selected from the group consisting of a square, a rectangular, a triangular, a hexagonal, an elliptical and a rhomboidal cross-section.

22. The flexible septal occluder of claim 19 wherein all of the strands of the first portion, second portion or central body portion comprise the same non-circular cross-section.

23. The flexible septal occluder of claim 19 wherein at least one of said first portion, second portion or central body portion comprises at least two strands, each of the two strands having different non-circular cross-sections.

24. A method for making a flexible septal occluder, comprising:
providing a first portion comprising a plurality of arms, said arms comprising a first wire and a second wire; and
providing a second portion comprising a plurality of arms, said arms comprising a first wire and a second wire; and
providing a central body portion comprising a first wire and a second wire,
wherein said first wire of said first portion, second portion, or central body portion is annealed at a first temperature and said second wire of said first portion, second portion, or central portion is annealed at a second temperature, and
assembling said first portion, second portion, and central body portion to form said flexible septal occluder.

25. A flexible septal occluder, comprising:
a first portion comprising a plurality of arms;
a second portion comprising a plurality of arms; and
a central body portion comprising a coil.

26. The flexible septal occluder of claim 25, wherein at least a portion of one said plurality of arms of said first portion or said second portion comprises a coil.
